(19) United States
(12) Patent Application Publication
(10) Pub. No.: US 2006/0106209 A1
(43) Pub. Date: May 18, 2006

(54) Novel process for the preparation of o-acylated glucose derivatives

(75) Inventors: Jean-Christophe Henrion, Pantin (FR); Michel Philippe, Wissous (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/331,124

(22) Filed: Jan. 13, 2006

Related U.S. Application Data
(63) Continuation of application No. 10/784,221, filed on Feb. 24, 2004.
(60) Provisional application No. 60/472,751, filed on May 23, 2003.

(30) Foreign Application Priority Data
Mar. 4, 2003 (FR) 0302635

(51) Int. Cl. C07H 13/02

(52) U.S. Cl. 536/119

(57) ABSTRACT

Process for the preparation of O-acylated glucose derivatives, in particular O-acylated predominantly in the 6 position, by reaction of a carboxylic acid with an alkyl haloformate, so as to form a mixed anhydride, and then reaction of the mixed anhydride with glucose.

### REFERENCE TO PRIOR APPLICATIONS

This application claims priority to U.S. provisional application 60/472,751 filed May 23, 2003, and to French patent application 0302635 filed Mar. 4, 2003, both incorporated herein by reference.

### FIELD OF THE INVENTION

The present invention relates to a novel process for the preparation of O-acylated glucose derivatives, in particular O-acylated predominantly in the 6 position. The product of the invention process also makes up a part of the invention, as does the preparation of cosmetic and dermatological compositions containing the product of the invention.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

O-Acylated glucose derivatives are generally already known in the prior art, which describes several syntheses thereof.

Several methods for the esterification of D-glucose with lauric acid have been described and compared in the journal “Die Starke”, No. 6, p. 181-189, in 1968, by Reinefeld et al. Provision was thus made, among agents for acylating glucose, to employ acid chlorides, such as lauroyl chloride, acid imidazolides, in particular of lauric acid, or mixed carboxylic-carbonic anhydrides.

It emerges from this publication that the method which makes it possible to obtain the highest yield is acylation using the acid chloride. With lauroyl chloride, a mixture of monoester and of diesters, with a yield of 49%, including 36% for the monoester, is obtained, for example. However, it is not always easy to have available the appropriate acid chloride. In the absence of industrial acid chloride, it is then necessary to use another method.

Acylation employing acid imidazolides results in a mixture of monoester and of diesters, with a total yield of 22% and a yield of 9% for the monoester alone, when lauric acid imidazolide is used.

This document also describes a process for the preparation of D-glucopyranose 6-laurate, in which, in a first stage, lauric acid is reacted with ethyl chloroformate, in the presence of triethanolamine, in benzene, so as to form a mixed anhydride, and then, in a second stage, the said mixed anhydride is reacted with D-glucose, in pyridine, at 80° C. The desired compound is thus obtained with a yield which can range from 21 to 28%, according to the relative amounts of the starting reactants. However, the reaction is carried out in benzene, a solvent which is difficult to use industrially as it is banned in some countries, or in pyridine under hot conditions. The other disadvantages related to this process are, first, the low yield and, secondly, the problems of purification related to the removal of the imidazole.

Furthermore, the acylation of glucose via the formation of a true anhydride results in the desired compounds with a total yield of 46% for the mixture of monoester and diesters and of 28% for the production of the monoester. This process brings about the formation of free fatty acids, which have to be removed in order to result in relatively pure final products. In point of fact, this removal may sometimes prove to be difficult, in view of the nature of the impurities; furthermore, there is generally a desire to avoid intermediate purification stages, which unnecessarily lengthen the process and which generate additional costs, this being incompatible with an industrial process.

It was found that, whatever the method envisaged, the acylating agent chosen and/or the proportion of each of the reactants, the acylation of glucose always resulted in the production of a mixture in which it was possible to identify D-glucopyranose 6-ester but also D-glucopyranose 1,6-diester and D-glucopyranose 2,6-diester as coexisting reaction products.

U.S. Pat. No. 5,498,708 also discloses a process for the preparation of polyol esters which consist in reacting an acid, for example a fatty acid, with an aryl or C₁₋₁₀ alkyl chloroformate in a 100% aqueous medium, so as to form an anhydride, and in then reacting the said anhydride with a polyol, so as to form the desired compound. The reaction is carried out in crushed ice and thus at a temperature close to 0C. It should be noted that the only chloroformate given as an example is ethyl chloroformate, presented in the description as more particularly preferred reactant. Here again, the operating conditions, in particular of handling in crushed ice, may prove to be difficult to implement industrially.

In addition, Patent EP 566 438 discloses a process for the preparation of D-maltose monoesters which consists, in a first step, in preparing a mixed anhydride, by reaction of a carboxylic acid and of an alkyl chloroformate in the presence of a base, and then, in a second step, in reacting the said mixed anhydride with D-maltose.

By virtue of this process, it is possible, surprisingly, to selectively acylate the 6′ position of D-maltose and not the 6 position carried by a glucose unit, the anomeric functional group of which is nevertheless free.

### SUMMARY OF THE INVENTION

The need thus remains to have available a novel route for the synthesis of O-acylated glucose derivatives which makes it possible to obtain these compounds rapidly and easily, at the industrial level, with a high yield of desired products. In point of fact, it had been found that, by using highly specific reactants, it is possible to achieve this aim, the reaction furthermore being carried out at a temperature close to ambient temperature, of the order of 20.25° C., which additionally makes it possible to avoid a heating stage.

### DETAILED DESCRIPTION OF THE PREFERRED EMBODIMENTS

One subject of the present invention is thus a process for the preparation of O-acylated glucose derivatives, in particular O-acylated predominantly (i.e., 100%, at least 98%, at least 95%, at least 90%, at least 85%, at least 80%, at least 75%, at least 70%, at least 65%, at least 60%, at least 65%, at least 60%, at least 55%, greater than 50%, at least 50%, etc) in the 6 position, of formula (I): 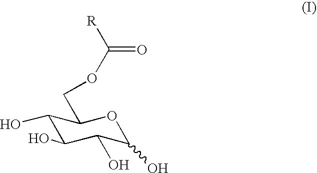
in which R is a saturated or unsaturated and linear or branched hydrocarbon chain comprising 7 to 21 carbon atoms, comprising the steps of:
in a first stage, preparing a mixed anhydride of formula (II): 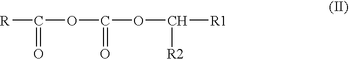
   in which R1 and R2 are, independently of one another, saturated or unsaturated and linear or branched hydrocarbon radicals comprising 1 to 20 carbon atoms, by reaction of a carboxylic acid of formula R—COOH with an alkyl haloformate of formula X—C(O)—O—CHR1R2, with X representing a halogen, preferably chlorine or bromine;
in a second stage, reacting the said mixed anhydride formed with glucose.

The process which is a subject-matter of the present invention makes it possible to prepare in particular O-acylated glucose derivatives, O-acylated predominantly in the 6 position, alone or as a mixture, which can be represented by the formula (I).

This is all the more surprising since, in contrast to what would have been expected by a person skilled in the art on reading EP 566 438 mentioned above, the reaction takes place here on the 6 position of the glucose, the anomeric functional group of which is free.

Furthermore, this regioselectivity for the 6 position of the glucose would also appear unexpected to a person skilled in the art as it would have been expected that the most reactive OH functional group of the glucose would be the anomeric hemiacetal OH.

Preferably, the R radical is a saturated or unsaturated and linear or branched hydrocarbon chain comprising 11 to 17 carbon atoms.

The acyl residue —COR can in particular be an octanoyl, decanoyl, dodecanoyl, myristoyl, hexadecanoyl, stearoyl, palmitoleoyl, oleoyl, linoleoyl or linolenoyl residue.

Mention may thus be made, without limitation, among the carboxylic acids capable of being employed to prepare the mixed anhydride of formula (II), of octanoic, decanoic, dodecanoic, myristic, hexadecanoic, stearic, oleic, linoleic or linolenic acid, and their mixtures.

Among the alkyl haloformates of formula X—C(O)—O—CHR1R2, use will more particularly be made of the compounds for which R1 and/or R2 are, independently of one another, saturated or unsaturated and linear or branched hydrocarbon radicals comprising 1 to 6 carbon atoms, in particular R1 and/or R2 are chosen from methyl or ethyl, and more particularly the compounds X—C(O)—O—CH(CH₃)₂, or isopropyl haloformates, and in particular isopropyl chloroformate.

The reaction scheme of the first stage of the process may be as follows:

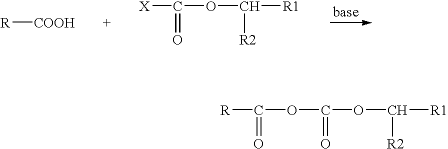

The reaction can be carried out in a reaction organic solvent, such as tetrahydrofuran, N-methylpyrrolidone, pyridine, toluene and their mixtures, preferably in toluene.

It can preferably be carried out under an inert atmosphere, for example nitrogen.

A base can be used to activate the carboxylic acid or else the corresponding carboxylate can be used directly; this base is preferably an organic base chosen in particular from triethylamine, pyridine, 4-dimethylaminopyridine, tributylamine, N-methylmorpholine and their mixtures, preferably triethylamine.

The reaction can be carried out at a temperature of −25° C. to +40° C., preferably −10° C. to +10° C., and for a time of 5 minutes to 5 hours, in particular of 30 minutes to 3 hours.

Preferably, 0.3 to 3 equivalents, preferably 0.5 to 1.5 equivalents, of carboxylic acid are reacted with 1 equivalent of halide.

In the second stage of the process, the esterification stage, the said mixed anhydride is reacted with glucose.

This second stage can optionally be carried out after filtering off the salts possibly formed during the first stage.

This second stage is preferably carried out in an organic solvent, which can be the same organic solvent as that of the first stage. This solvent can therefore be tetrahydrofuran, N-methylpyrrolidone, pyridine, toluene and their mixtures, preferably pyridine.

Preferably, the mixed anhydride is dissolved in the said organic solvent before the reaction.

Preferably, the glucose is dissolved beforehand in a solvent, such as pyridine, N-methylpyrrolidone and/or dimethylacetamide, preferably pyridine.

Preferably, 0.5 to 1.5, in particular 0.9 to 1.1 and better still 1 equivalent(s) of mixed anhydride is/are reacted with 3 equivalents of glucose.

Use is preferably made, according to the invention, of an excess of glucose, for example at least 3 equivalents of glucose, with respect to the acid or to the mixture of acids reacted in the first stage.

One advantage of the invention is that this second stage of the process can be carried out at a temperature close to ambient temperature, for example at a temperature of between 10° C. and 40° C., preferably 15° C. to 30° C. and better still 18° C. to 25° C.

The reaction can be carried out for a time of 1 to 15 hours, in particular of 2 to 8 hours.

After the end of the reaction, the solvents can be separated from the desired compound, for example by evaporation, centrifuging or filtration.

The resulting product can be purified by any known means, such as distillation, chromatography on a column of silica gel, precipitation and/or extraction, for example with a water/organic solvents mixture.

The process according to the invention thus makes it possible to prepare, in a way which can be implemented industrially, O-acylated glucose derivatives, O-acylated predominantly in the 6 position, alone or as a mixture. In particular, the following compounds can be prepared according to this process: 6-O-octadeca-9,12-dienoyl-D-glucopyranose; 6-O-octadeca-9-enoyl-D-glucopyranose; 6-O-octadecanoyl-D-glucopyranose; 6-O-hexadecanoyl-D-glucopyranose; and their mixtures. In particular, the glucose esters of vitamin F can be prepared by virtue of this process.

For the record, vitamin F is considered to be generally composed (% by weight):
of 75 to 80% by weight of linoleic acid, and
of 10 to 15% by weight of oleic acid,
of 4 to 8% by weight of palmitic acid,
of 0.5 to 3% by weight of stearic acid, and
of 0 to 10% by weight of one or more other acids chosen from lauric, myristic, arachidic, behenic, lauroleic, myristoleic, palmitoleic and linolenic acids.

The result of this is that the invention can make it possible to prepare, by esterification of vitamin F, a product which is composed of a mixture of different esters, resulting from the presence of the different acids, formed during this reaction.

It has been found that, generally, with the process according to the present invention, glucose was esterified mainly in the 6 position and possibly in the 3 position and/or in other positions.

The two stages of the invention reaction scheme can be accomplished in any manner available to one of ordinary skill in the art. Moreover, the two stages can be accomplished in one pot, if desired, or intermediate separation, isolation, purification, etc. can be accomplished in between stages.

The product(s) of the invention process can optionally be purified by any applicable methodology, and may be used as a component in, e.g., a cosmetic and/or dermatological composition comprising, for example, a physiologically acceptable medium. Simple addition, mixture, etc. (“combination”) of the product(s) with other components of such compositions is well within the skill of the ordinary artisan in view of this disclosure.

### EXAMPLE

### Preparation of the Glucose Ester of Vitamin F (Predominantly Ester in the 6 Position)

18.7 g of isopropyl chloroformate, as a 0.15M solution in toluene (153 ml), are run into a 1 litre reactor rendered inert with nitrogen. A mixture of 38.9 g of vitamin F and 15.5 g of triethylamine, dissolved beforehand in 45 ml of toluene, is added, under an inert atmosphere and at 0° C.; the mixture is left stirring at 20° C. for 1 hour and then the salts formed are filtered off in order to obtain a solution.

100 g of D-glucose are dissolved under warm conditions in 1 litre of pyridiine in a 2.5 litre reactor and the preceding solution is added thereto, under an inert atmosphere, at ambient temperature (20° C.). The mixture is left stirring at 20° C. for 4 hours.

The reaction medium is evaporated to dryness under vacuum in order to remove the pyridine, the paste obtained is then extracted (water/organic solvent mixture) and the organic phase recovered is dried, filtered and evaporated.

47.5 g of a yellow paste of vitamin F ester are obtained, which ester includes 67% of monoesters (mixture) in the 6 position.

The ¹H and ¹³C NMR spectra, (d₆-DMSO), 200 MHz, are in accordance with the expected structure.

The above written description of the invention provides a manner and process of making and using it such that any person skilled in this art is enabled to make and use the same, this enablement being provided in particular for the subject matter of the appended claims, which make up a part of the original description and including a process for the preparation of O-acylated glucose derivatives, in particular O-acylated predominantly in the 6 position, of formula (I): 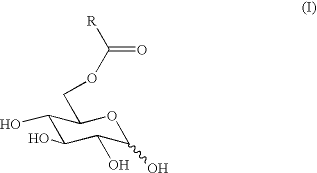
in which R is a saturated or unsaturated and linear or branched hydrocarbon chain comprising 7 to 21 carbon atoms, comprising the steps:
in a first stage, of preparing a mixed anhydride of formula (II): 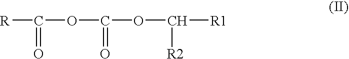
in which R1 and R2 are, independently of one another, saturated or unsaturated and linear or branched hydrocarbon radicals comprising 1 to 20 carbon atoms, by reaction of a carboxylic acid of formula R—COOH with an alkyl haloformate of formula X—C(O)—O—CHR1R2, with X representing a halogen, preferably chlorine or bromine;
in a second stage, of reacting the said mixed anhydride formed with glucose. Also fully described and enabled is a process for the preparation of an O-acylated glucose derivative, comprising:
preparing a mixed anhydride of formula (II): 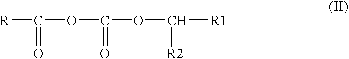
in which R1 and R2 are, independently of one another, saturated or unsaturated and linear or branched hydrocarbon radicals comprising 1 to 20 carbon atoms and R is a saturated or unsaturated, linear or branched hydrocarbon chain comprising 7 to 21 carbon atoms, by reaction of a carboxylic acid of formula R—COOH with an alkyl haloformate of formula X—C(O)—O—CHR1R2, with X representing a halogen, preferably chlorine or bromine; and
reacting said mixed anhydride with glucose.

As used above, the phrases “selected from the group consisting of,” “chosen from,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out. Where the term “between” is used in a range the endpoints are included; for example, the phrase “between 10° C. and 40° C.” includes both 10° C. and 40° C.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for the preparation of an O-acylated glucose derivative in which the O-acylated glucose derivative prepared is O-acylated at least 50% in the 6 position, comprising:
preparing a mixed anhydride of formula (II): 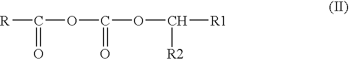
in which R1 and R2 are, independently of one another, saturated or unsaturated and linear or branched hydrocarbon radicals comprising 1 to 20 carbon atoms and R is a saturated or unsaturated, linear or branched hydrocarbon chain comprising 7 to 21 carbon atoms,
by reaction of a carboxylic acid of formula R—COOH with an alkyl haloformate of formula X—C(O)—O—CHR1R2, with X representing halogen; and
reacting said mixed anhydride with glucose.

2. The process according to claim 1, said derivative having formula (I): 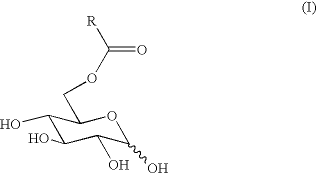
in which R is a saturated or unsaturated, linear or branched hydrocarbon chain comprising 7 to 21 carbon atoms.

3. The process according to claim 1, in which the acyl residue —COR in formula II is a residue selected from the group consisting of octanoyl, decanoyl, dodecanoyl, myristoyl, hexadecanoyl, stearoyl, palmitoleoyl, oleoyl, linoleoyl and linolenoyl residues.

4. The process according to claim 1, in which the alkyl haloformate is selected from the group consisting of compounds for which R1 and/or R2 are, independently of one another, saturated, linear hydrocarbon radicals comprising 1 to 6 carbon atoms.

5. The process according to claim 1, in which R1 and/or R2 are selected from the group consisting of methyl and ethyl.

6. The process according to claim 1, in which R1 and/or R2 are selected from the group consisting of the compounds X—C(O)—O—CH(CH₃)₂.

7. The process according to claim 1, in the alkyl haloformate is an isopropyl haloformate.

8. The process according to claim 1, in which the alkyl haloformate is isopropyl chloroformate.

9. The process according to claim 1, in which the mixed anhydride is prepared in an organic solvent.

10. The process according to claim 1, in which the mixed anhydride is prepared in an organic solvent selected from the group consisting of tetrahydrofuran, N-methylpyrrolidone, pyridine, toluene and mixtures thereof.

11. The process according to claim 1, in which the mixed anhydride is prepared in toluene.

12. The process according to claim 1, in which the mixed anhydride is prepared at a temperature of −25° C. to +40° C. and for a time of 5 minutes to 5 hours.

13. The process according to claim 1, in which the mixed anhydride is prepared at a temperature of −10° C. to +10° C. for a time of 5 minutes to 5 hours.

14. The process according to claim 1, in which the mixed anhydride is prepared at a temperature of −25° C. to +40° C. and for a time of 30 minutes to 3 hours.

15. The process according to claim 1, in which the mixed anhydride is prepared at a temperature of −10° C. to +10° C. for a time of 30 minutes to 3 hours.

16. The process according to claim 1, in which the reaction of said mixed anhydride with glucose is carried out in an organic solvent.

17. The process according to claim 1, in which the reaction of said mixed anhydride with glucose is carried out in an organic solvent selected from the group consisting of tetrahydrofuran, N-methylpyrrolidone, pyridine, toluene and mixtures thereof.

18. The process according to claim 1, in which the reaction of said mixed anhydride with glucose is carried out in pyridine.

19. The process according to claim 1, in which the reaction of said mixed anhydride with glucose is carried out at a temperature of 10° C.-40° C.

20. The process according to claim 1, in which the reaction of said mixed anhydride with glucose is carried out at a temperature of 15° C.-30° C.

21. The process according to claim 1, in which the reaction of said mixed anhydride with glucose is carried out at a temperature of 18° C.-25° C.

22. The process according to claim 1, in which the reaction of said mixed anhydride with glucose is carried out for a time of 1 to 15 hours.

23. The process according to claim 1, in which the reaction of said mixed anhydride with glucose is carried out for a time of 2 to 8 hours.

24. The process according to claim 1, wherein said O-acylated glucose derivative is selected from the group consisting of 6-O-octadeca-9,12-dienoyl-D-glucopyranose, 6-O-octadeca-9-enoyl-D-glucopyranose, 6-O-octadecanoyl-D-glucopyranose, 6-O-hexadecanoyl-D-glucopyranose and mixtures thereof.

25. The process according to claim 1, wherein said O-acylated glucose derivative is selected from the group consisting of glucose esters of vitamin F and mixtures thereof.

26. The process according to claim 2, wherein said O-acylated glucose derivative is selected from the group consisting of 6-O-octadeca-9,12-dienoyl-D-glucopyranose, 6-O-octadeca-9-enoyl-D-glucopyranose, 6-O-octadecanoyl-D-glucopyranose, 6-O-hexadecanoyl-D-glucopyranose and mixtures thereof.

27. The process according to claim 2, wherein said O-acylated glucose derivative is selected from the group consisting of glucose esters of vitamin F and mixtures thereof.

28. The process according to claim 1, further comprising:
optionally purifying the product of the reaction of said mixed anhydride with glucose to produce a purified product, and
combining said optionally purified product with a physiologically acceptable medium to provide a cosmetic or dermatological composition.

29. The process according to claim 25, further comprising:
optionally purifying the product of the reaction of said mixed anhydride with glucose to produce a purified product, and
combining said optionally purified product with a physiologically acceptable medium to provide a cosmetic or dermatological composition.

30. The process according to claim 1, wherein R is a saturated or unsaturated, linear or branched hydrocarbon chain comprising 11 to 17 carbon atoms.

31. The process according to claim 2, wherein R is a saturated or unsaturated, linear or branched hydrocarbon chain comprising 11 to 17 carbon atoms.
